(19) United States
(12) Patent Application Publication
(10) Pub. No.: US 2025/0040970 A1
(43) Pub. Date: Feb. 6, 2025

(54) BONE PLATE SYSTEM

(71) Applicant: Medline Industries, LP, Northfield, IL (US)

(72) Inventor: Ryan Niver, Glenview, IL (US)

(21) Appl. No.: 18/229,985

(22) Filed: Aug. 3, 2023

(51) Int. Cl. A61B 17/80; A61B 17/86

(52) U.S. Cl. CPC A61B 17/8052; A61B 17/8605

(57) ABSTRACT

A bone plate system can include at least one screw including a superelastic material. The system can further include a bone plate. A bone fixation method can include disposing a bone plate over bone and inserting at least one screw including superelastic material through the bone plate.

### FIELD

The disclosure relates to orthopedic appliances and methods pertaining to such appliances, and in particular systems and methods for fusing one or more of a bone fracture and a joint.

### BACKGROUND

Orthopedic bone compression systems can be used to compress fractures and joints. Some known bone compression systems include one or more bone plates and screws that can be fixed to bone segments across a fracture or joint fusion area. It is thought that a bone fracture can heal more effectively when subject to some degree of compressive loading. Conventional bone plate systems, however, typically are constructed of rigid materials such as titanium, which tends to inhibit compression.

Provided herein are bone plate systems and bone fixation methods intended to provide dynamic fixation. The bone plate system includes at least two screws and at least one bone plate. The bone plate generally includes at least two screw-receiving structures respectively defining at least two holes. At least one of the screws comprises a superelastic material. The plate is configured such that a superelastic screw can exert a dynamic biasing force against at least a portion of a receiving structure thereby when the bone plate is under tension between the at least two screws. This dynamic biasing force can be at an angle normal or oblique to the major axis of the screw to thereby permit a degree of loading of the fracture and/or joint.

In one form, the bone plate can include one or more holes formed to receive a screw in an oblique or normal orientation relative to at least one other screw through the bone plate. Alternatively, one or more of the screw-receiving structures can comprise an eccentric portion sized to engage a head of a screw and bias the plate laterally via camming action upon advancing the head of the screw against the eccentric portion. The eccentric portion can be a ramp surface extending about an insertion axis of the hole of a receiving structure. The bone plate can include one or both of these tensioning features. In either case, the lateral biasing can cause tension in the bone plate, this tension causing corresponding compression of the inferior bone segments.

A bone fixation method generally comprises disposing at least one bone plate over one or more of a joint between inferior bones or a fracture in an inferior fusion area and inserting at least two screws through respective holes in the bone plate and screwing the screws into bone, at least one of the screws comprising a superelastic material and the plate configured with a tensioning feature as described above. A kit may comprise a container, such as a sterile box, containing a bone plate and at least one superelasic screw, and optionally other components useful in an orthopedic procedure such as drill bits.

### BRIEF DESCRIPTION OF THE DRAWINGS

FIG. 1 is a representational view of one embodiment of a bone plate system including a bone plate, three screws formed of titanium, and two screws composed of superelastic material;

FIG. 2 is a side elevation view of one of superelastic screws of the system shown in FIG. 1;

FIGS. 2′, 3, and 3′ are side elevation views of useful alternative superelastic screws;

FIG. 4 is a perspective view of the bone plate of the system shown in FIG. 1;

FIG. 5 is a top plan view of the bone plate shown in FIG. 1;

FIG. 6 is a bottom plan view of the bone plate shown in FIG. 1;

FIG. 7 is a side elevation view of the bone plate shown in FIG. 1;

FIG. 8 is a perspective view of a bone structure including the bone plate secured to a plurality of inferior bones, showing an oblique screw in hidden lines;

FIG. 9 is a side view of the bone structure shown in FIG. 8;

FIG. 10 is a perspective view of a kit including a box containing the bone plate system shown in FIG. 1;

FIG. 11 is a perspective view of an alternative bone plate system having a bone plate, one screw formed of titanium, and one screw composed of a superelastic material;

FIG. 12 is a top plan view of the bone plate of the system of FIG. 11;

FIG. 13 is a bottom plan view of the bone plate shown in FIG. 11;

FIG. 14 is a side elevation view of the bone plate shown in FIG. 11;

FIG. 15 is a perspective view of a bone structure including the bone plate system of FIG. 11 secured to a plurality of inferior bones; and

FIG. 16 is a perspective view of an alternative bone plate system including a bone plate, three titanium screws, and two superelastic screws.

### DETAILED DESCRIPTION

A bone plate system can generally include any suitable number of screws and any number of bone plates suitable for the intended use of the system. A system will include at least one screw comprising a superelastic material and at least one bone plate including a screw-receiving structure defining a hole sized to receive the screw.

The system can include at least first and second screws and at least one bone plate, the plate including at least first and second screw-receiving structures respectively defining first and second holes respectively sized to receive the first and second screws. At least one screw of the first and second screws can comprise a superelastic material, such that when the bone plate is under tension with the at least one screw received within a hole, the at least one screw exerts a dynamic biasing force against at least a portion of the receiving structure defining the hole. In some forms, the first screw comprises a superelastic material and the second screw comprises another material. In other forms, the second screw comprises a superelastic material and the first screw comprises another material. In yet other forms, both the first and second screws comprise superelastic material. A superelastic screw can exert dynamic biasing force at any angle relative to an axis of the screw, such as an angle normal or oblique to an axis for the screw. Some bone plates will be sized for and include openings for third or fourth screws or additional screws, some or all of which can be composed of superelastic material or of a material that is not superelastic.

The superelastic material as provided herein can generally include any biocompatible superelastic material such as a superelastic metal alloy or polymer. The superelastic material can also be considered a shape memory material. Examples of superelastic metal alloys include a nitinol alloy, which is a family of nickel-titanium alloys, and beta-titanium alloys such as TiNbZrHfSn. An example of superelastic polymer is polyether ether ketone (PEEK). Other screws useful in the systems described herein, and the bone plates described herein, can be made of any suitable biocompatible materials other than superelastic materials, such as titanium or titanium alloy.

The superelastic screw and the other screws described herein can optionally be a solid screw not including any cannulation or cavity in the shaft of the screw. Alternatively, one or more of the superelastic screw and the other screws provided herein can have cannulation or cavities in a shaft portion of the screw. Cannulated and non-cannulated screws can be used in combination.

A screw comprising a superelastic material as provided herein is generally a non-compression screw. A compression screw includes one or more cavities along a length of a shaft of the screw, and the one more cavities permit axial expansion of the screw when the screw is under tension such that a superelastic material in the screw applies an axial compressive force in response to the expansion.

Any screw as provided herein can be a lag screw or a fully threaded screw. A lag screw has an unthreaded portion proximal a head of the screw and a threaded portion proximal a tip of the screw. A fully threaded screw has threads extending an entire length of a shaft, or substantially an entire length of a shaft of the screw from the tip to the head of the screw. The heads of lag and fully threaded screws optionally can have a locking structure such as threading that engages threading formed in a screw-receiving structure of a bone plate. Alternatively, the heads of lag and fully threaded screws can have a smooth peripheral surface lacking a locking structure for engagement with a smooth surface of a screw-receiving structure.

A kit generally includes a container housing one or more bone plate systems. The container can include any one or more of a box, a bag, an envelope, and vacuum seal packaging. A kit preferably includes a container including at least a sterile interior that houses sterile components of one or more bone plate systems. The kit may include other components useful in an orthopedic procedure such as drill bits, screws of different sizes, guides and guide wires, and so forth.

The bone fixation method generally includes applying one or more bone plate systems provided herein to a surgical site. This generally comprises disposing at least one bone plate over one or more of a joint between inferior bones or a fracture in an inferior fusion area, and in any appropriate order inserting a first screw through a first hole defined by a first screw-receiving structure of a bone plate and screwing the first screw into bone, and inserting a second screw through a second hole defined by a second screw-receiving structure of a bone plate and screwing the second screw into bone, wherein at least one screw of the first and second screws comprises a superelastic material. Third and fourth screws and any additional screws may be inserted where applicable and in any order appropriate. Any one or more of the third, fourth, and any additional screws can optionally comprise a superelastic material. The method may include drilling one or more pilot holes into the bone.

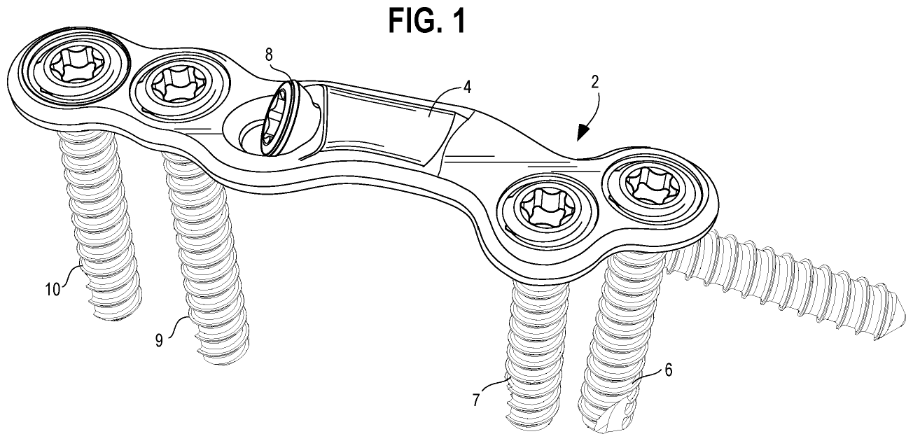

The bone plate system 2 illustrated in FIG. 1 includes a bone plate 4, three titanium screws 6, 7, 8, and two screws 9, 10 composed of superelastic material. One of the titanium screws 8 is disposed through a central portion of the plate at an angle relatively oblique to the other four screws.

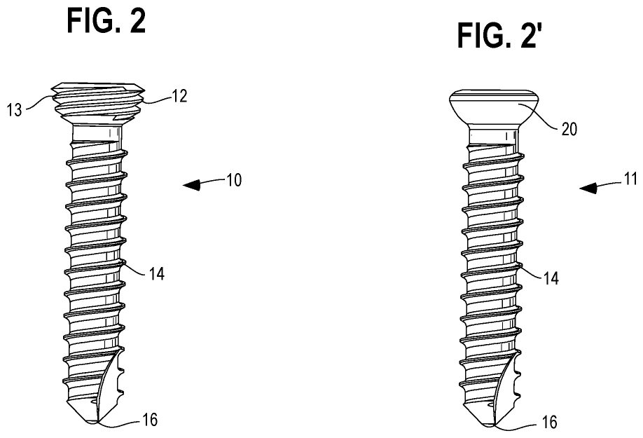

As seen in FIG. 2, screw 10 includes a head 12, shaft 14, and tip 16. The head 12 includes threaded portion 13 for interfacing with a threaded screw-receiving structure in the bone plate. The shaft 14 is fully threaded and is solid without cannulation or cavities. FIG. 2′ illustrates an alternative screw 11 having the same structure as the screw 10 in FIG. 2 except the head 20 of screw 11 is smooth and without threading. FIG. 3 illustrates an alternative lag screw 18 that includes a shaft 22 that is without cannulation or cavities and has unthreaded lag region 19 adjacent threaded region 21 that extends from a medial region of the shaft to the tip 16. The head 20 of the screw 18 is smooth and without threading. FIG. 3′ illustrates yet another alternative lag screw 23 having a structure that is the same as the screw 18 in FIG. 3 except that the head 12 of screw 23 includes threaded portion 13. While screws 10, 11, 18 and 23 comprise superelastic material, any of screws 10, 11, 18 and 23 can be formed of any useful material and be used singly, along with one or other screws, or in any useful combination.

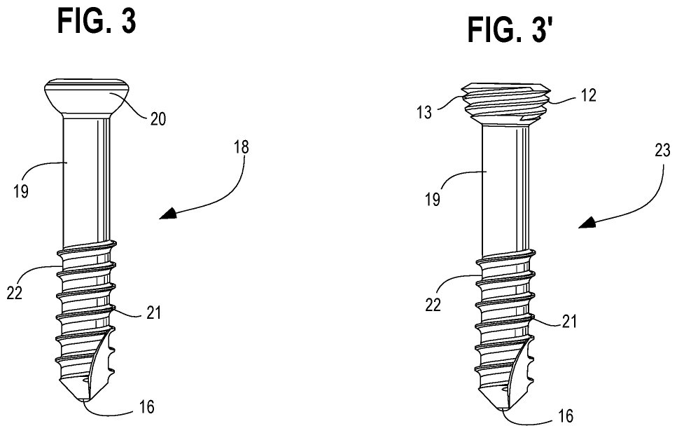

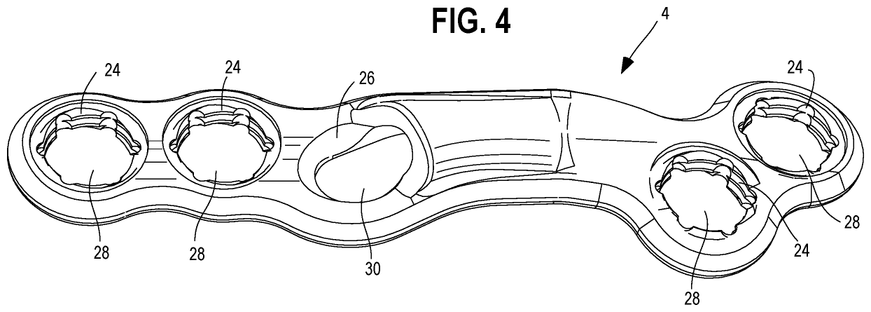

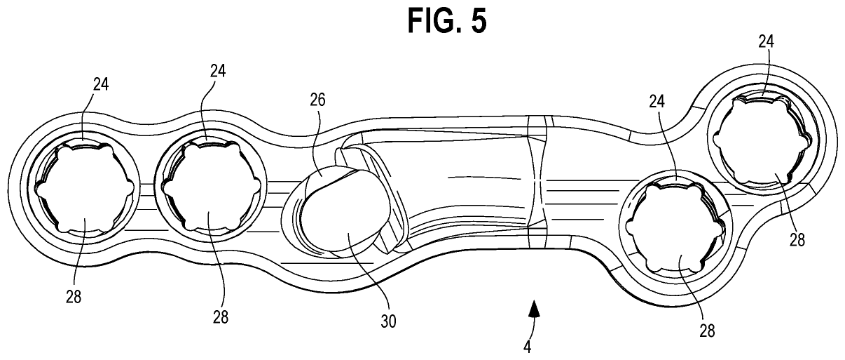

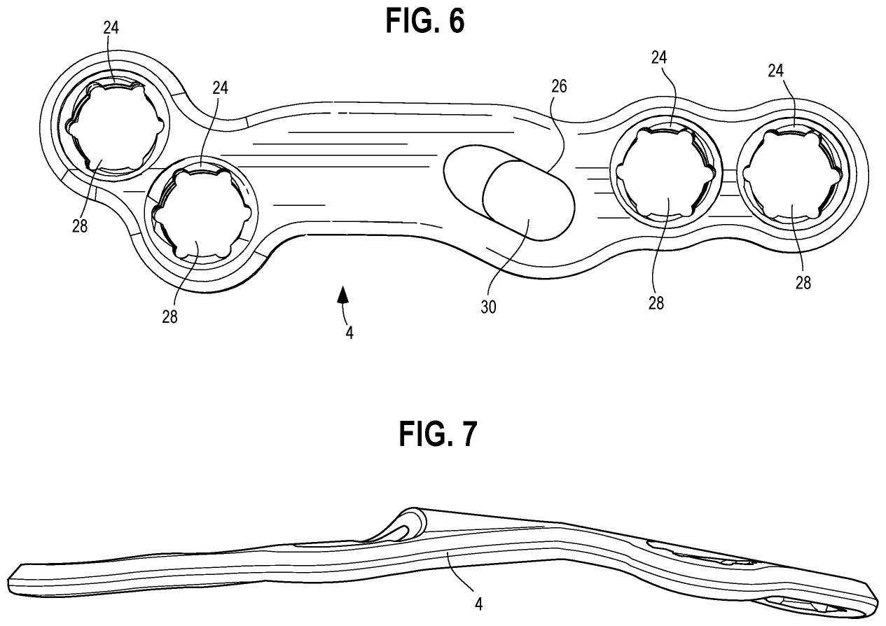

As further seen in FIG. 4-7, the bone plate 4 includes four threaded screw-receiving structures 24 each defining a hole 28 (FIGS. 4-6). The threaded screw-receiving structures engage threaded screw head. The bone plate also includes an unthreaded screw-receiving structure 26 defining a hole 30 formed to receive a screw in an oblique orientation relative to at least one other screw disposed through the other holes 28 in the plate 4. Alternatively, one or more screw receiving structures can define holes to receive screws in normal orientation relative to at least one other screw disposed through a bone plate. In some forms, one or more screws oriented normally relative to other screws through a bone plate can comprise a superelastic material.

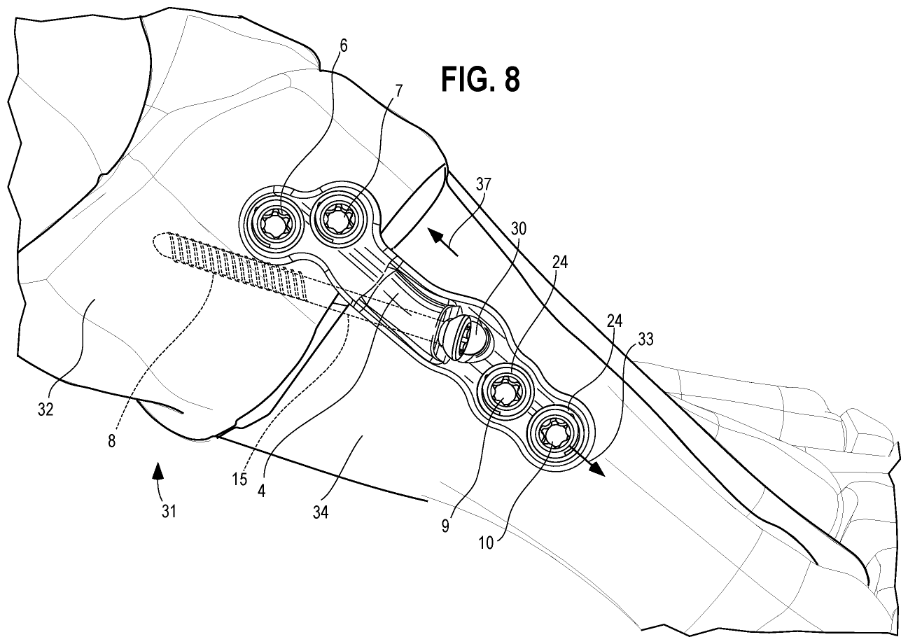

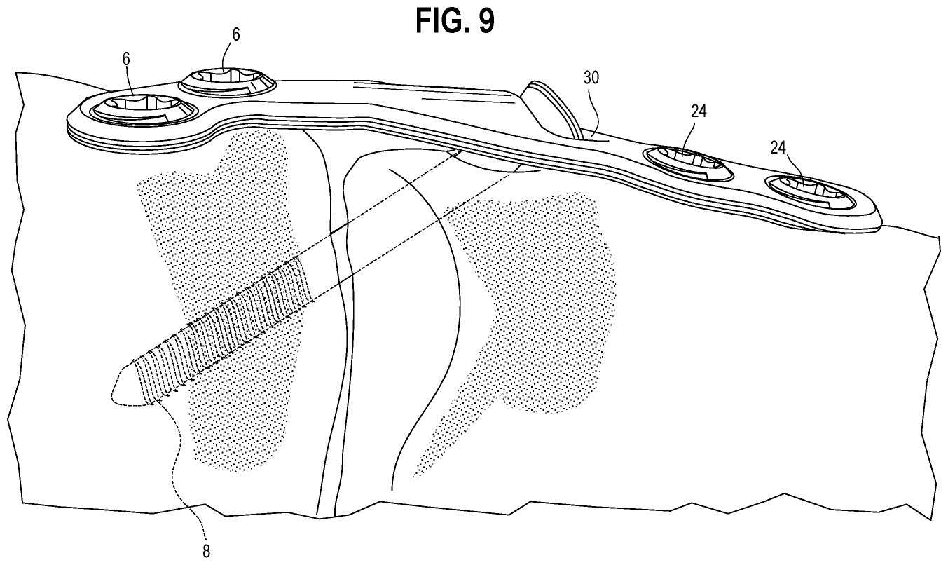

As seen in FIGS. 8 and 9, the bone structure 31 comprises a plurality of inferior bone segments 32, 34. Two screws 9, 10 composed of superelastic material have been inserted through holes in the bone plate 4 and screwed into bone segment 34. A screw 8 formed of titanium has been inserted through a hole 30 in the bone plate and screwed into bones 34 and 32. The screw 8 is in an oblique orientation relative to both screws 9, 10 including superelastic material. The shaft 15 of the screw 8 reaches across bone segments 32, 34. Additional screws 6, 7 formed of titanium have been inserted through holes formed in the bone plate and screwed into bone 32. Via this configuration, screw 8 exerts tension through the plate 4 generally in direction 37, which causes at least one of screws 9, 10 to exert a dynamic biasing force generally in direction 33 against at least a portion of a respective receiving structure 24 forming the hole through which the screws 9, 10 are inserted. Alternatively, screw 8 can include a superelastic material and exert a dynamic biasing force against at least a portion of screw receiving structure 26 when the plate is under tension between at least one of screws 9, 10 and screw 8, where screws 6, 7, 9, 10 can each individually be composed of superelastic or any other useful material.

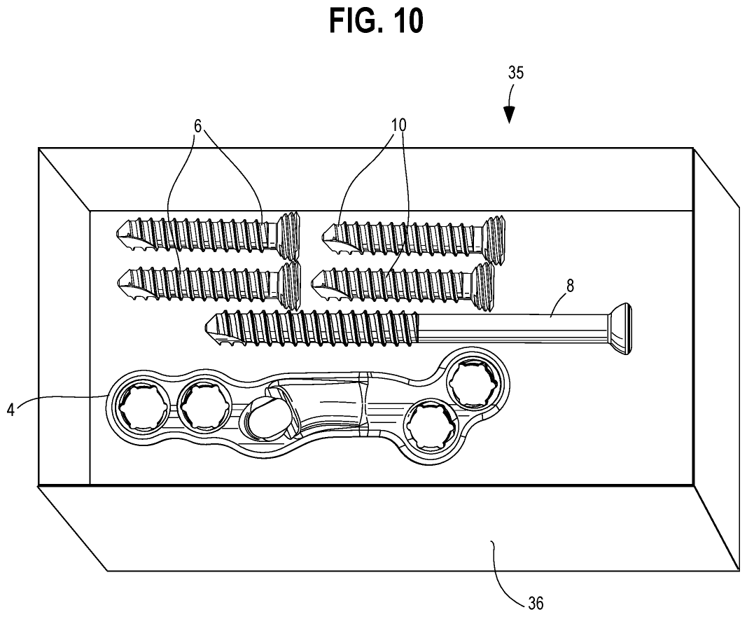

The kit 35 shown in FIG. 10 includes a box 36 and the bone plate system 2, with bone plate 4 and screws 6, 7, 8, 9, 10.

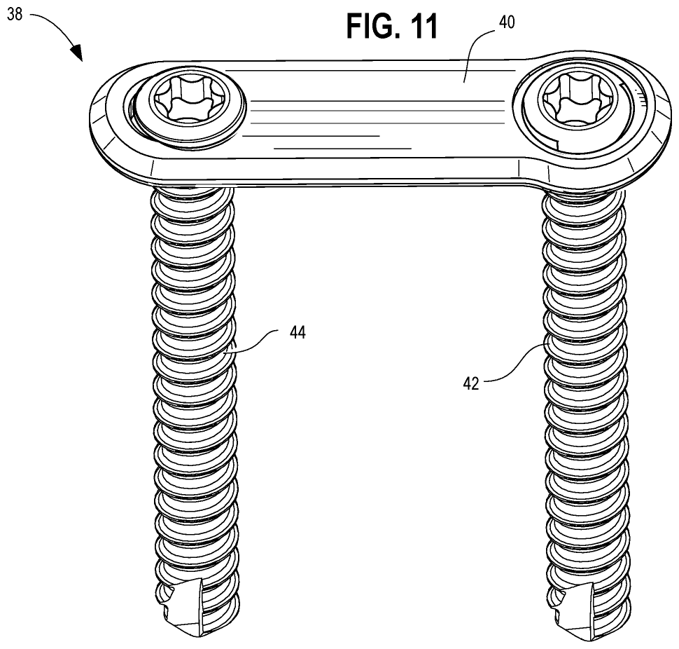

The alternative bone plate system 38 shown in FIG. 11 includes a bone plate 40, a screw formed of titanium 44, and a screw composed of superelastic material 42. The shaft of the superelastic screw 42 is solid without cannulation or cavities. This system may be provided with a box in the form of a kit (not shown) as with kit 35 shown in FIG. 10. Screws 42 and 44 are shown as fully threaded but one or both of screws 42, 44 could be provided as a lag screw. In an alternative form, screw 44 could be composed of a superelastic material and screw 42 could be formed of titanium. In yet another form, both screws 42 and 44 could composed of a superelastic material.

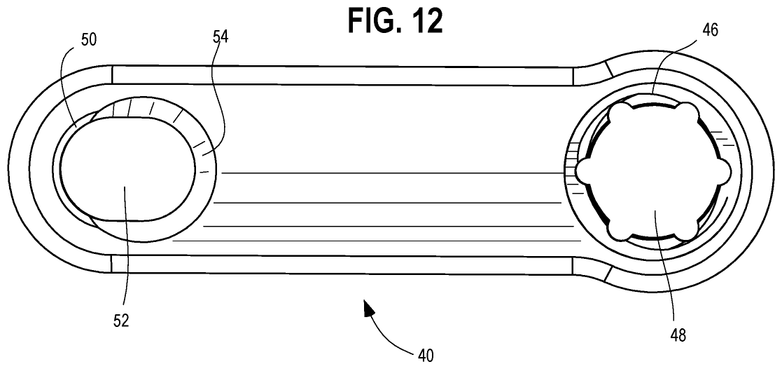

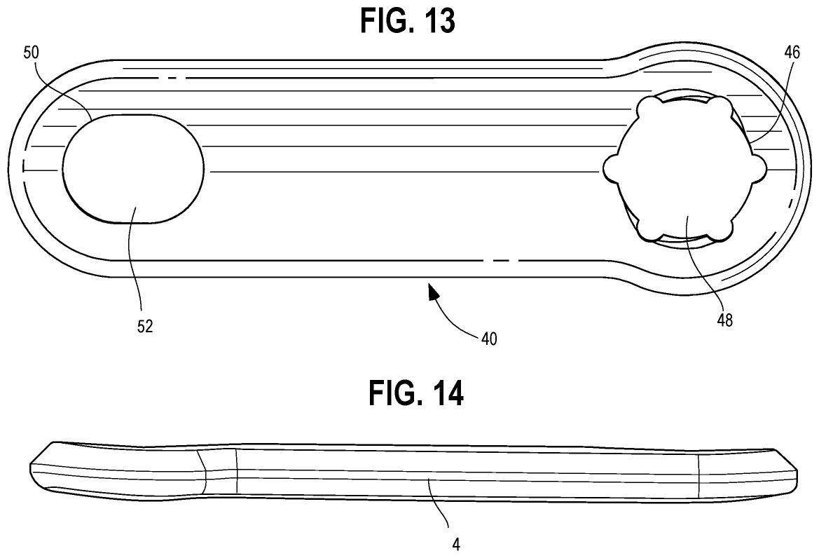

As seen in FIGS. 12-14, the bone plate 40 includes a screw-receiving structure 46 defining a hole 48 sized to receive a shaft of the superelastic screw 42. The bone plate further includes a screw-receiving structure 50 defining a hole 52 sized to receive a shaft of the screw 44 formed of titanium. The screw-receiving structure 50 includes a ramped surface 54 extending around a portion of the hole 52.

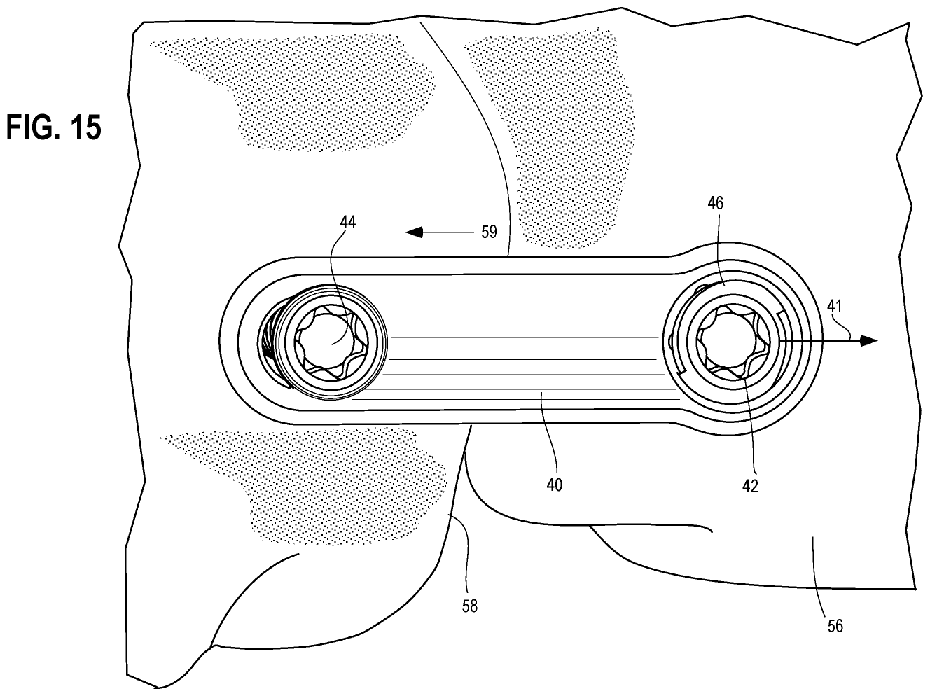

FIG. 15 illustrates an embodiment of application of the bone plate system of FIG. 11 to a plurality of inferior bone segments 56, 58. The superelastic screw 42 has been inserted through the hole in the bone plate 40 and screwed into bone 56. The titanium screw 44 has been inserted through the hole corresponding to screw-receiving structure 50 including the ramped surface. The screw 44 has been screwed into bone 58 such that the head of the screw engages the ramped surface and applies tension through the plate 40 and laterally biases the plate in direction 59 via camming action. This has placed the bone plate 40 under tension between screw 42 and screw 44 such that screw 42 exerts a dynamic biasing force in direction 41 against a portion of the receiving structure 46 forming the hole through which the screw 42 is inserted. In an alternative form, screw 44 can be composed of superelastic material and screw 42 can be formed of titanium such that screw 44 exerts a dynamic biasing force against a portion of the receiving structure with which it is engaged when the plate is under tension between the screws 42, 44. In another alternative form where both screws 42 and 44 are composed of superelastic material both screws 42, 44 can exert dynamic biasing forces against respective receiving structures through with which the screws are engaged when the plate is under tension between the screws 42, 44.

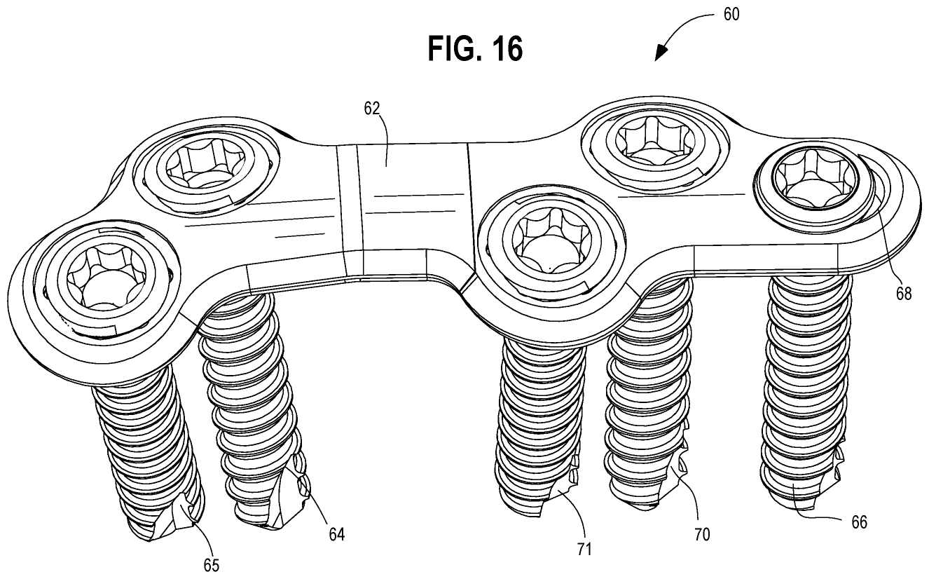

The alternative bone plate system 60 shown in FIG. 16 includes a bone plate 62, three screws 66, 70, 71 formed of titanium, and two screws 63, 64 including superelastic material. A screw-receiving structure 68 at one of end of the bone plate includes a ramp surface like that shown in FIG. 11. The ramp surface can be engaged by a head of the screw 66 disposed through the corresponding hole to laterally bias the plate via camming action.

The superelastic screws 64, 65 can be inserted through the bone plate 62 and screwed into bone. A titanium screw 66 can be inserted through the hole formed by the screw-receiving structure 68 and screwed into bone such that the head of the screw 66 engages the ramp surface and biases the plate 62 laterally via camming action. Tension applied in the bone plate 62 between the screws 64, 65 and the screw 66 can dynamically compress a joint or fracture area. Additional screws 70, 71 formed of titanium can be inserted through the intermediate region of the bone plate 62 and screwed into bone to further secure the bone plate to bone.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A bone plate system comprising:
at least first and second screws,
a bone plate including at least first and second screw-receiving structures respectively defining first and second holes, the first and second holes being respectively sized to receive the first and second screws, at least one screw of the first and second screws comprising a superelastic material, and
when the bone plate is under tension with the first and second screws respectively received within the first and second holes, the at least one screw exerts a dynamic biasing force against at least a portion of the first or second receiving structure.

2. The bone plate system according to claim 1, wherein the first screw comprises a superelastic material and exerts the dynamic biasing force against the first receiving structure at an angle normal or oblique to an axis for the first screw.

3. The bone plate system according to claim 1, wherein the second hole is formed to receive the second screw in an oblique or normal orientation relative to the first screw.

4. The bone plate system according to claim 3, further comprising at least a third screw, the bone plate further comprising at least a third screw-receiving structure defining a third hole sized to receive the third screw, the second hole being disposed in an intermediate region of the bone plate between the first hole and the third hole.

5. The bone plate system according to claim 4, further comprising at least fourth and fifth screws, the bone plate further comprising at least fourth and fifth screw-receiving structures respectively defining fourth and fifth holes respectively sized to receive the fourth and fifth screws, the fourth hole being proximal to the first hole, the fifth hole being proximal to the third hole, and at least the first and fourth screws comprise superelastic material and, when the bone plate is under tension with the first and fourth screws respectively received within the first and fourth holes, at least one of the first and fourth screws respectively exerts a dynamic biasing force against at least a portion of the respective first or fourth receiving structure.

6. The bone plate system according to claim 5, wherein the first screw exerts the dynamic biasing force at an angle normal or oblique to an axis for the first screw, and the fourth screw exerts the dynamic biasing force at an angle normal or oblique to an axis for the fourth screw.

7. The bone plate system according to claim 1, wherein the second screw-receiving structure comprises an eccentric portion sized to engage a head of the second screw and to bias the plate laterally via camming action.

8. The bone plate system according to claim 7, wherein the eccentric portion comprises a ramp surface extending about an insertion axis of the second hole.

9. The bone plate system according to claim 1, wherein the first screw comprises nitinol.

10. The bone plate system according to claim 9, wherein the first screw is solid.

11. The bone plate system according to claim 10, wherein the first screw is a lag screw having an unthreaded portion adjacent a head of the screw, the unthreaded portion sized to engage the first receiving structure to thereby screw exert the dynamic biasing force.

12. The bone plate system according to claim 5, wherein the first and fourth screws comprise nitinol.

13. The bone plate system according to claim 1, wherein the bone plate comprises titanium.

14. A kit comprising container housing the bone plate system according to claim 1.

15. A bone fixation method comprising:
disposing a bone plate over one or more of a joint between inferior bones or a fracture in an inferior fusion area, the bone plate including at least first and second screw-receiving structures respectively defining first and second holes,
inserting a first screw through the first hole and screwing the first screw into bone, and
inserting a second screw through the second hole and screwing the second screw into bone, the second hole receiving the second screw in an oblique or normal orientation relative to the first screw with the second screw reaching across the one or more of the joint or the fracture,
at least one screw of the first and second screws comprising a superelastic material, and tension applied through the bone plate between the first and second screws causing the at least one screw to exert a dynamic biasing force against at least a portion of the first or second receiving structure.

16. The bone fixation method according to claim 15, wherein the first screw comprises a superelastic material and exerts the dynamic biasing force at an angle normal or oblique to an axis for the first screw.

17. The bone fixation method according to claim 15, wherein the bone plate further comprises at least a third screw-receiving structure defining a third hole, the second hole being disposed in an intermediate region of the bone plate between the first hole and the third hole, and
the method further comprises inserting a third screw through the third hole and screwing the third screw into bone, the first and third screws being screwed into bone on opposite sides of the one or more of the joint or the fracture.

18. The bone fixation method according to claim 17, wherein the bone plate further comprises at least fourth and fifth screw-receiving structures respectively defining fourth and fifth holes, the fourth hole being proximal to the first hole, the fifth hole being proximal to the third hole, and
the method further comprises inserting a fourth screw through the fourth hole screwing the fourth screw into bone, inserting a fifth screw through the fifth hole and screwing the fifth screw into bone, wherein the first and fourth screws comprise a superelastic material, and the tension applied through the bone plate causes at least one of the first and fourth screws to exert a dynamic biasing force against at least a portion of the respective first and fourth receiving structures.

19. The bone fixation method according to claim 18, wherein the first and fourth screws exert the dynamic biasing forces each at an angle respectively normal or oblique to axes for the first and fourth screws.

20. The bone fixation method according to claim 15, wherein the first screw comprises nitinol.

21. The bone fixation method according to claim 18, wherein the first and fourth screws comprise nitinol.

22. The bone fixation method according to claim 15, wherein the first screw comprises a superelastic material and the method further comprises drilling first and second bore holes into bone, the first bore hole being drilled at a position to be proximal the portion of the first receiving structure where the first screw exerts the dynamic biasing force, and the second bore hole being drilled at a position to correspond to the second hole, the first screw being screwed into the first bore, and the second screw being screwed into the second bore.

23. A bone fixation method comprising:
disposing a bone plate over one or more of a joint between inferior bones or a fracture in an inferior fusion area, the bone plate including at least first and second screw-receiving structures respectively defining first and second holes, the second screw-receiving structure comprising an eccentric portion proximal the second hole,
inserting a first screw through the first hole and screwing the first screw into bone, and
inserting the second screw through the second hole and screwing the second screw into bone, a head of the second screw engaging the eccentric portion via camming action and dynamically compressing the one or more of the joint or the fracture as the second screw is advanced into the bone, at least one screw of the first and second screws comprising a superelastic material, and tension applied through the bone plate between the first and second screws causing the at least one screw screw to exert a dynamic biasing force against at least a portion of the first or second screw-receiving structure.

24. The bone fixation method according to claim 23, wherein the first screw comprises a superelastic material and exerts the dynamic biasing force at an angle normal or oblique to an axis for the first screw.

25. The bone fixation method according to claim 23, wherein the eccentric portion comprises a ramp surface extending about an insertion axis of the second hole.

26. The bone fixation method according to claim 23, wherein the first screw comprises nitinol.

27. The bone fixation method according to claim 27, wherein the first screw is solid.

28. The bone fixation method according to claim 23, wherein the first screw comprises a superelastic material and the method further comprises drilling first and second bore holes into bone, the first bore hole being drilled at a position to be proximal the portion of the first receiving structure where the first screw exerts the dynamic biasing force, and the second bore hole being drilled at a position to correspond to the second hole, the first screw being screwed into the first bore, and the second screw being screwed into the second bore.

29. A bone plate system comprising:
a screw comprising a superelastic material, and
a bone plate including a screw-receiving structure defining a hole sized to receive the screw.
